(19) United States
(12) Patent Application Publication
(10) Pub. No.: US 2026/0272731 A1
(43) Pub. Date: Sep. 17, 2026

(54) BELT SUSPENDED FULLY DETACHABLE CLOTH DIAPER WITH COMPATIBLE BELTED GARMENTS

(71) Applicant: Chelsea Marian McAmis, Chattanooga, TN (US)

(72) Inventor: Chelsea Marian McAmis, Chattanooga, TN (US)

(21) Appl. No.: 19/670,628

(22) Filed: May 7, 2026

Related U.S. Application Data
(63) Continuation of application No. 18/512,317, filed on Nov. 17, 2023.
Application No. 18/512,317 is a continuation of application No. 17/346,892, filed on Jun. 14, 2021, now abandoned.

(51) Int. Cl. A61F 13/56

(52) U.S. Cl. CPC A61F 13/5638

(57) ABSTRACT

A garment system for managing incontinence comprises an absorbent article and one or more carrier garments. The absorbent article includes a liquid-impervious outer material, an inner-facing material, and at least one absorbent unit. The absorbent article contains no belt, waistband wings, waist-encircling structure, or crotch panel, rendering it incapable of independent wear. One or more carrier garments, selected from a waist belt, crotchless pants, crotchless shorts, a crotchless bodysuit, a skirt, a dress, or a shirt, each include a hip-surrounding portion and outward-facing fasteners. When connected, the carrier is sandwiched between the wearer's lower torso and the absorbent article. Discrete front and rear fastener sets allow independent detachment from either end, enabling absorbent article replacement without removing the carrier. The absorbent article is interchangeable among multiple carrier types while each carrier remains continuously worn.

### CROSS-REFERENCE TO RELATED APPLICATION

This application claims the benefit of priority to U.S. Non-Provisional patent application Ser. No. 18/512,317, filed Nov. 17, 2023, entitled “BELT SUSPENDED FULLY DETACHABLE CLOTH DIAPER WITH COMPATIBLE BELTED GARMENTS,” and U.S. Non-Provisional patent application Ser. No. 17/346,892, filed Jun. 14, 2021, entitled “BELT SUSPENDED FULLY DETACHABLE CLOTH DIAPER WITH COMPATIBLE BELTED GARMENTS”, both of which are incorporated herein by reference their entirety.

### FIELD

This application relates to absorbent garments, specifically diapers, worn by infants, children, and incontinent individuals.

### BACKGROUND

It is common practice in Western society to place infants and young children in absorbent garments, typically diapers, at all times for the containment of bodily excretions. However, the practice of infant potty training, also known as elimination communication (EC), is widely practiced around the globe, particularly in areas with restricted access to disposable diapers. This practice involves learning and responding to an infant's cues that indicate the infant's need to eliminate and offering the infant an appropriate receptacle for urine or feces. The practice can begin as young as birth and leads to a toilet-independent child at an age typically younger than infants who are diapered full-time and then potty trained at a later age. The practice of EC has gained popularity in Western society in addition to being practiced in other parts of the world. Many caregivers who practice EC use conventional diapers in addition to offering the toilet to their child.

The most common type of diaper currently used for infants and children is a disposable diaper having a longitudinal direction and a lateral direction and comprising of a front part, a rear part, an intermediate crotch part, and an absorbent unit for liquids. Typically, the diaper has a waist belt section on the rear-most edge of the rear part and on the front-most edge of the front part, with waist straps extending laterally from the waist of the rear portion. The diaper-wearer is placed in the dorsal recumbent position for diapering, the rear portion is placed below the wearer's buttocks, and the rear waist belt is aligned with the wearer's natural waist. The front portion is then drawn upward between the wearer's legs so that the front waist belt also aligns with the wearer's waist, and then the rear waist straps are brought around the wearer's waist and connected to the front part via fastening means, typically hook and loop.

Disposable diapers and cloth diapers of this kind have the following drawbacks:

After achieving mobility, many children are no longer content to lie on their backs for diaper changes, frequently rolling away from their caregiver and hindering the diaper-changing process. Additionally, many adult incontinent individuals have difficulty being diapered in the dorsal recumbent position.

Conventional diapers are inconvenient for caregivers practicing EC or potty training, as they pose a barrier to the toilet that must be removed and replaced, possibly along with other layers of clothing, during the process of offering the toilet to the child.

Belt diapers are a form of diaper that aim to solve these issues. Such diapers comprise a hip belt that forms a hip opening of the diaper which is closed in the peripheral direction, and which can be typically opened and closed on top of itself on at least one location using first and second fastening devices. Such a diaper further comprises a front part, a rear part, an intermediate crotch part, and an absorbent unit for liquids, wherein the diaper main part can be detachably fixed, at the longitudinal end of its front part or rear part, to the hip belt via third fastening devices, such that the main diaper part can be pulled between the wearer's legs after application of the hip belt, and detachably fix the free longitudinal end of the diaper main part to the hip belt.

Embodiments of belt diapers previously described have at least one or more of the following disadvantages:

If the belt is permanently attached to the front of the diaper, the diaper can only be opened from the rear and pulled forward to offer the toilet to the wearer. In this position, the diaper obstructs a male wearer's penis, making it difficult for a caregiver to assist the wearer to aim his urine into the toilet. Examples of this type include U.S. Pat. No. 6,764,479 to Kusibojoska et al. (2004), U.S. application 20080097369 to Melander (2008), “Flaparaps” sold on bornready.eu, among others.

If the belt is permanently attached to the rear of the diaper, the diaper can only be opened from the front and pulled backward to offer the toilet to the wearer. If the wearer has a bowel movement with this arrangement, the diaper impedes access to the wearer's rear end for cleaning. Examples of this type include U.S. Pat. No. 8,821,467 to Minella (2014), U.S. application 20100036336 to Bogdanova et al. (2010), U.S. Pat. No. 6,764,479 to Kusibojoska et al. (2004), U.S. Pat. No. 6,955,668 to Almberg et al. (2005), U.S. Pat. No. 9,211,220 to Schambon (2015), “Drop-Flap Diaper” sold on windelmanufaktur.com, “Hold-off sets” sold on Emilino.de, among others.

If the belt is permanently attached in any position or must be attached to the diaper before the belt can be placed on the wearer, then both diaper and belt must be removed every time the wearer wets or soils the diaper, causing inconvenience to the caregiver. Examples of this type include U.S. Pat. No. 5,135,522 to Fahrenkrug et al. (1992), U.S. application 20020091369 to Hansson (2002), U.S. Pat. No. 4,909,802 to Ahr et al. (1990), U.S. Pat. No. 6,342,050 to Rönnberg et al. et al. (2002), U.S. application 20070299418 to Vartiainen (2007), among others.

The diaper requires hook and loop closures to fasten to the belt, a method of fastening that often results in a shorter product lifespan than other closure methods. Examples of this type include U.S. Pat. No. 5,549,593 to Ygge et al. (1996), U.S. Pat. No. 8,449,518 to Allison-Rogers (2010), U.S. Pat. No. 5,971,970 to Carlbark et al. (1999), U.S. Pat. No. 9,554,952 to Rönnberg et al. (2017), among others.

The diaper requires central fasteners along a central longitudinal axis in the front and/or rear of the diaper in addition to lateral fasteners in the front and rear to be successfully attached to the belt, causing inconvenience to the caregiver. Examples of this type include U.S. Pat. No. 6,334,858 to Rönnberg et al. (2002), “Holding Trousers” sold on windelmanufaktur. com, among others.

The diaper is disposable; disposable diapers are costly for caregivers and place a strain on the environment. Examples of this type include U.S. Pat. No. 7,794,442 to Roehrl et al. (2010), U.S. Pat. No. 8,449,518 to Allison-Rogers (2010), W.I.P.O. publication 1991008725 to New et al. (1990), U.S. Pat. No. 6,432,099 to Rönnberg (2002), Spanish publication 2328478 to Rohrl et al. (2009), among others.

The diaper can only be attached to an article of clothing with sleeves for the legs, which means the diaper cannot be used by wearers in warmer climates or seasons. An example of this type includes Chinese publication 203885723 to Xuechenfang (2014).

Neither the belt nor the diaper has means of covering the wearer's legs, leaving the legs bare unless they are covered with outerwear such as pants, leg warmers, a bodysuit, or other clothing, which inconveniences the caregiver during diaper changes and when offering the toilet to the wearer. Examples of this type include U.S. Pat Nos. 6,334,858 and 6,432,099 to Rönnberg et al. (2002), U.S. Pat. No. 5,549,593 to Ygge et al. (1996), U.S. application 20110184372 to Östlin et al (2011), U.S. Pat. No. 7,794,442 to Roehrl et al. (2010), U.S. Pat. No. 8,449,518 to Allison-Rogers (2010), W.I.P.O. publication 1991008725 to New et al. (1990), U.S. Pat. No. 5,971,970 to Carlbark et al. (1999), U.S. Pat. No. 9,554,952 to RRönnberg nnberg et al. (2017), U.S. application 20060167432 to Sigari (2006), among others.

The diaper is designed for use in adult incontinent individuals and does not adequately reflect the needs of caregivers practicing EC or potty training with their infants and children. An example of this type includes U.S. application 20050192555 to Thomas (2005).

The belt is intended to wrap around the waist of the diaper after the diaper is placed on the wearer to inhibit the diaper's removal by the wearer or to aid in securing it; such belt arrangements obstruct the practice of EC. An example of this type is U.S. application 20140068839 to Steele et al. (2014).

The belt is an elastic ring meant to hold up a separate diaper without a means of fastening the diaper to the belt. Such belt arrangements do not securely hold the diaper and are prone to leaks. Examples of this type include “rECtangle diaper covers” sold on ecwear.com, “Li'l Baby Chaps” sold on ecwear.com, “Windfall 6Pcs/Pack Adjustable Baby Diaper Fastener Nappy Fixed Belt Infant Cloth Holder” sold on Walmart.com, “Adjustable Nappy Diaper belt, prefold belt, cloth nappy belt, elimination communication clothing, nappy free baby clothing, EC gear, baby” sold by LittleBunnyBear on Etsy.com, “Baby Diaper Fastener 4 Hook Safety Nappy Fixing Belt” sold on us.patpat.com, among others.

### Notes on Construction

The use of the terms “a”, “an”, “the” and similar terms in the context of describing the invention are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. The terms “substantially”, “generally” and other words of degree are relative modifiers intended to indicate permissible variation from the characteristic so modified. The use of such terms in describing a physical or functional characteristic of the invention is not intended to limit such characteristic to the absolute value which the term modifies, but rather to provide an approximation of the value of such physical or functional characteristic.

Terms concerning attachments, coupling and the like, such as “connected” and “interconnected”, refer to a relationship wherein structures are secured or attached to one another either directly or indirectly through intervening structures, as well as both moveable and rigid attachments or relationships, unless specified herein or clearly indicated by context. The term “operatively connected” is such an attachment, coupling or connection that allows the pertinent structures to operate as intended by virtue of that relationship.

The use of any and all examples or exemplary language (e.g., “such as” and “preferably”) herein is intended merely to better illuminate the invention and the preferred embodiment thereof, and not to place a limitation on the scope of the invention. Nothing in the specification should be construed as indicating any element as essential to the practice of the invention unless so stated with specificity.

### SUMMARY OF THE EMBODIMENTS

[symbol]

### BRIEF DESCRIPTION OF THE DRAWINGS

Further advantages of the invention are apparent by reference to the detailed description when considered in conjunction with the figures, which are not to scale so as to more clearly show the details, wherein like reference numerals represent like elements throughout the several views, and wherein:

FIG. 1 illustrates an interior view of an absorbent article (diaper) when the diaper is laid flat;

FIG. 2 illustrates an exterior view of the absorbent article (diaper) when the diaper is laid flat;

FIG. 3 illustrates a fully detachable waist belt when it is opened and laid flat;

FIG. 4 illustrates a perspective view of the waist belt when it is closed;

FIG. 5 illustrates the diaper partially attached to the waist belt at the front fastening devices for both the waist belt and the diaper so that the rear part of the diaper hangs freely;

FIG. 6 illustrates the diaper partially attached to the waist belt at the rear fastening devices for both the waist belt and the diaper so that the front part of the diaper hangs freely;

FIG. 7 illustrates a rear view of the diaper fully attached to the waist belt, which is positioned on a wearer;

FIG. 8 illustrates a front view of the diaper fully attached to the waist belt, which is positioned on a wearer;

FIG. 9 illustrates a perspective view of the front of a pant garment with waist belt;

FIG. 10 illustrates a perspective view of the rear of the pant garment with waist belt;

FIG. 11 illustrates a front view of the diaper fully attached to the waist belt of the pant garment, which is positioned on a wearer;

FIG. 12 illustrates a rear view of the diaper fully attached to the waist belt of the pant garment, which is positioned on a wearer;

FIG. 13 illustrates a perspective view of the front of a skirt garment with waist belt;

FIG. 14 illustrates the skirt garment with a portion of the skirt material lifted up to reveal the positions of the fastening devices, which are hidden underneath the skirt fabric when the garment is positioned on a wearer;

FIG. 15 illustrates a rear view of the diaper fully attached to the waist belt of the skirt garment, which is positioned on a wearer;

FIG. 16 illustrates a pattern shape that is used to cut out the inner facing material and liquid impervious top sheet of the diaper in one embodiment;

FIG. 17 illustrates a pattern shape that is used for crafting the pant garment in one embodiment;

FIG. 18 illustrates a front view of the diaper fully attached to a shirt garment with waist belt, which is positioned on a wearer;

FIG. 19 illustrates a rear view of the diaper fully attached to a bodysuit garment with waist belt, shirt portion, and sleeves for legs, which is positioned on a wearer; and

FIG. 20 illustrates a perspective view of a dress garment with waist belt, skirt, and shirt portion; part of the skirt of the dress garment is lifted to reveal fastening devices.

### DETAILED DESCRIPTION

It is the aim of the present application to solve the disadvantages discussed in the background. Thus, some embodiments provide a caregiver a choice between unfastening a diaper from the front or the rear in the process of offering the toilet to a wearer. Some embodiments enable the diaper to be changed without removing a compatible garment from the wearer in the process. Some embodiments of the diaper will be compatible with a belt that leaves the wearer's legs bare as well as a pant garment that covers the wearer's legs, enabling the diaper to be worn in any climate or season. Some embodiments will employ snap closures instead of hook and loop, a sturdier and longer-lasting closure method, particularly for washable garments. Some embodiments will use four closures in order to successfully attach the diaper to the waist belt of the compatible garment, two fasteners arranged laterally in the front and two arranged laterally in the rear. Some embodiments are washable and reusable, making the embodiment a cheaper choice for caregivers and less of a burden on the environment. Some embodiments will be specifically designed for use on infants with caregivers practicing EC. These and other benefits of one or more aspects will become apparent from a consideration of the ensuing description and accompanying drawings.

### Description of the Absorbent Article

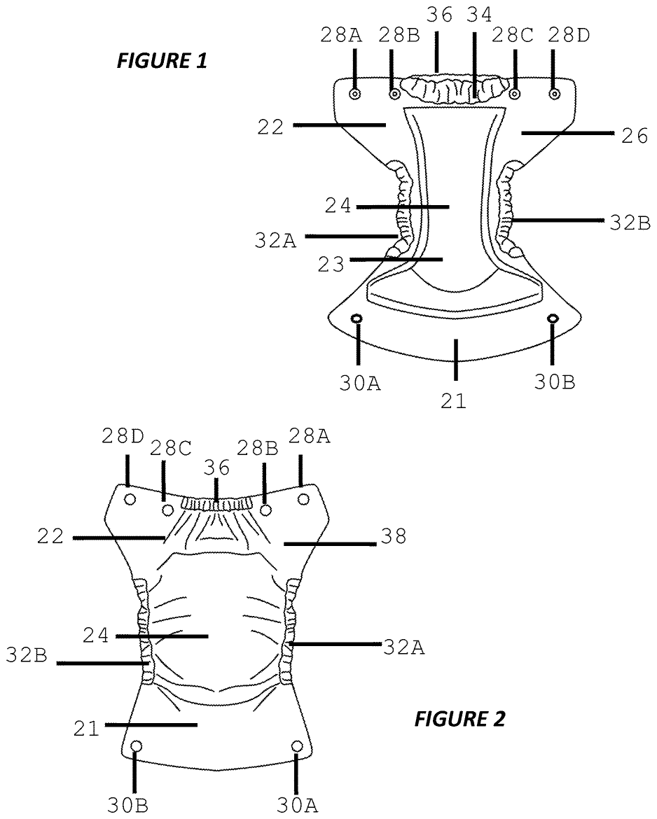

FIG. 1 shows a view of the interior of an embodiment of an absorbent article, most typically referred to as a diaper or nappy, when the diaper is laid flat. FIG. 2 shows a view of the exterior of the diaper when the diaper is laid flat. Both FIGS. 1 and 2 indicate a front part 21, a rear part 22, and an intermediate crotch part 24. An outer layer of the diaper shown in FIG. 2 is a liquid impervious top sheet 38. In this embodiment, the top sheet 38 is cut from a length of polyurethane laminate fabric (PUL), a breathable, water-repellent fabric often used in the manufacture of cloth diapers. Other materials suitable for this purpose would include wool, plastic, certain kinds of fleece, among others. The fabric for this layer is cut into a fitted diaper shape, a pattern for which is shown in FIG. 16. FIG. 16 indicates that in this embodiment, the front part 21 of the diaper will be cut narrower than the rear part 22, with the intermediate crotch part 24 tapering between the two widths. An inner facing material 26 shown in FIG. 1 is cut in the same pattern shape as shown in FIG. 16. In this embodiment, the inner facing material 26 is cut from a length of fleece and sewn to the liquid impervious top sheet 38. Other suitable materials for the inner facing material would be suede, microfleece, terrycloth, birdseye cotton, diaper twill, among others.

As shown in FIG. 1, an absorbent unit 23 is sewn to the inner facing material 26. In this embodiment, the absorbent unit is made of folded layers of 100% cotton birdseye fabric. This embodiment uses eight to ten layers of birdseye cotton; however, different materials would be suitable for the absorbent unit in different numbers of layers (for example, diaper twill, terrycloth, absorbent microfiber, among others).

The diaper shown in FIGS. 1 and 2 is given further shaping in this embodiment by adding lateral elastic elements 32A and 32B as well as front and rear elastic elements 34, 36 in inner facing material 26 and the liquid impervious top sheet 38. Elastic ribbon is used for this embodiment, but other forms of elastic, such as fold-over elastic, are suitable. As seen in FIG. 1, sewing separate elastic elements 34, 36 into the rear part of the inner facing material 26 and the rear part of the liquid impervious top sheet 38 provides the diaper of this embodiment with a pocket between the inner facing material 26 and the top sheet 38 into which additional absorbent units could be inserted to increase the diaper's absorbency. In one embodiment, this is achieved by sewing the absorbent unit 23 to a liquid impervious inner facing material 26 and then removing the inner facing material directly beneath the absorbent unit so that liquid may pass through the absorbent unit 23 and into the pocket between the inner facing material 26 and top sheet 38. In other embodiments, the inner facing material 26 and the absorbent unit 23 are both made from absorbent material, rendering the process of cutting the inner facing material unnecessary. In other embodiments, the absorbent unit 23 is fully detached from the inner facing material 26 and inserted directly into the pocket formed by the inner facing material 26 and top sheet 38. Other methods exist for crafting such a pocket.

The embodiment of FIGS. 1 and 2 also has four rear fastening devices 28A-28D and two front fastening devices 30A and 30B. In this embodiment, the fastening devices are snaps with male snaps located at the lateral edges of the rear part 22 of the diaper and female snaps located at the lateral edges of the front part 21 of the diaper. Although snaps are used in this embodiment, other closure devices, such as buttons or hook and eye, may be used instead.

### Description of the Waist Belt

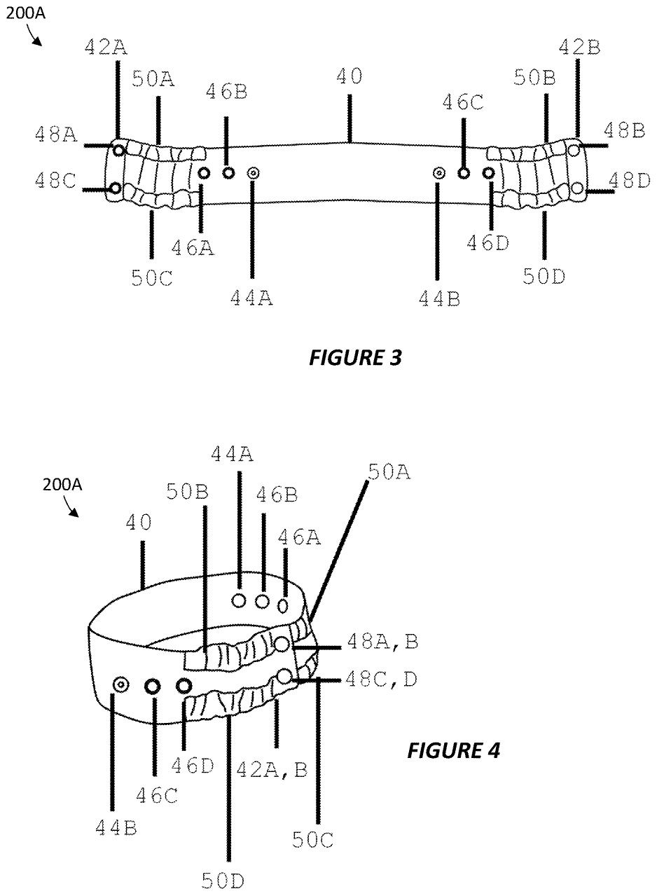

FIG. 3 shows a detached waist belt that is open and laid flat. FIG. 4 shows the same waist belt that has been closed using belt closure devices 48A-48D. As can be seen in FIG. 3, rear parts 42A and 42B of the belt are divided in half when the belt closure devices 48A-48D are released. A front part 40 of the belt shown in FIG. 3 is then in the center of the belt when laid flat. In FIG. 4, the two halves of the rear part 42A and 42B have been joined by the belt closure devices 48A-48D, which have been fastened. This places the rear parts 42A and 42B directly behind the front part 40.

In this embodiment, the waist belt is comprised of fleece, but other materials such as wool or PUL could be used. The belt is shaped by cutting a rectangle of fleece of dimensions 10.16 centimeters by 53.34 centimeters, although other sized rectangles would be appropriate for embodiments intended for larger or smaller individuals. This rectangle is folded on itself along its length to form a rectangle measuring 5.08 centimeters by 53.34 centimeters, which is then sewn along the raw edges. Elastic elements 50A-50D, shown in both FIG. 3 and FIG. 4, are then added to provide shaping.

The belt of FIGS. 3 and 4 also has front fastening devices 44A and 44B and rear fastening devices 46A-46D. These devices 44A, 44B, and 46A-46D are snaps in this embodiment, with the front snaps being male snaps and the rear snaps being female snaps. The belt closure devices 48A-48D shown in FIGS. 3 and 4 are also made of snaps, with two male snaps 48B and 48D on one half of the rear part 42B and two belt closure devices 48A and 48C (e.g., female snaps) on the other half of the rear part 42A so that the male and female snaps may be brought together to connect the two halves as shown in FIG. 4. Other closure devices besides snaps could also be used in other embodiments.

### Compatibility of Absorbent Article and Waist Belt

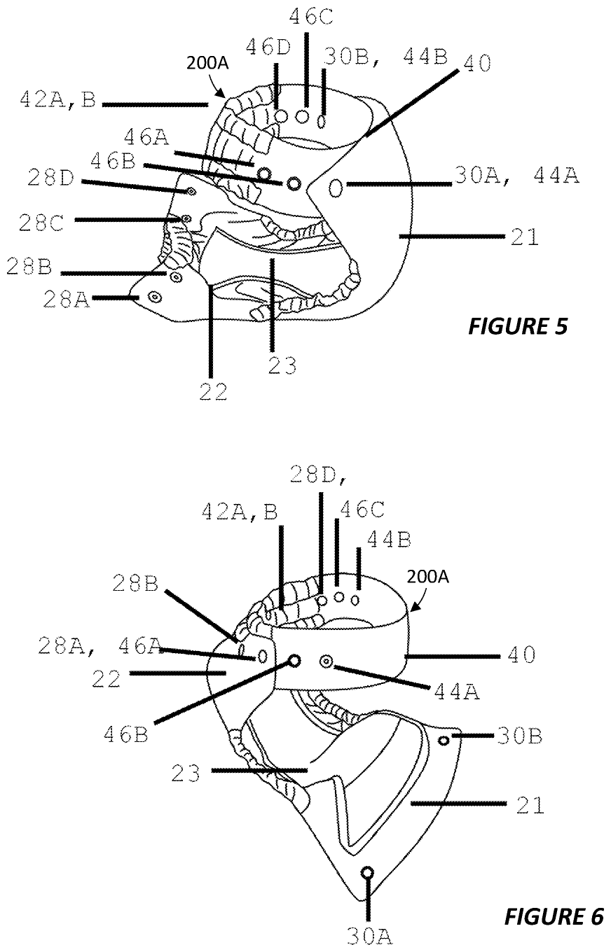

FIG. 5 shows how the waist belt can be fastened to the diaper by connecting the front fastening devices 44A, 44B on the belt to the front fastening devices 30A and 30B, respectively. The belt is shown as being closed in FIG. 5 and the rear fastening devices 28A-28D are not connected to any of the rear fastening devices 46A-46D of the belt. In this embodiment, the front fastening devices 44A, 44B of the belt are male snaps, which are connected to female snaps that are the front fastening devices 30A and 30B of the diaper. In this position, the front part 21 of the diaper is snugly resting against the front part 40 of the belt while the rear part 22 of the diaper hangs freely from the rear parts 42A and 42B of the belt, allowing the absorbent unit 23 to be visible.

FIG. 6 shows how the waist belt can be fastened to the diaper by connecting two of the rear fastening devices 46A and 46D on the belt to two of the rear fastening devices 28A and 28D on the diaper, respectively. The belt is shown as being closed in FIG. 6 and the front fastening devices 30A and 30B are not connected to the front fastening devices 44A, 44B of the belt. In this embodiment, the rear fastening devices 46A-46D of the belt are female snaps, only two of which need be connected to two of the male snaps that are the rear fastening devices 28A and 28D of the diaper. Two of the rear fastening devices 28B, 28C, which are snaps in this case, are not snapped to any other snap; additionally, two of the rear fastening devices 46B and 46C of the belt (e.g., snaps) are not snapped to any other snap. This is due to the fact that for this embodiment, only two snaps are required to satisfactorily connect the rear part 22 of the diaper to the rear parts 42A and 42B of the belt. In this position, the rear part 22 of the diaper is snugly resting against the rear parts 42A and 42B of the belt while the front part 21 of the diaper hangs freely from the front part 40 of the belt, allowing the absorbent unit 23 to be visible.

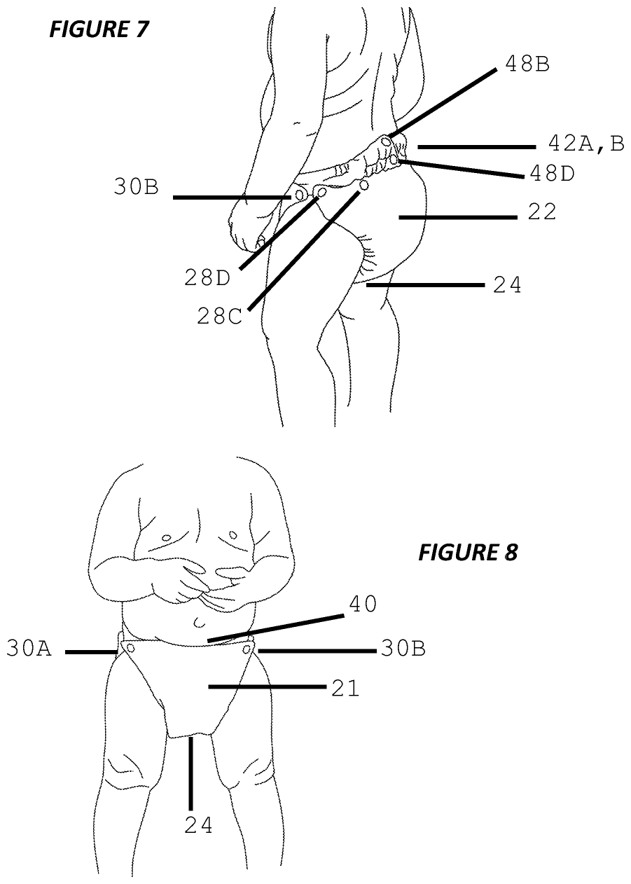

FIG. 7 illustrates a rear view of the diaper fully attached to the waist belt, which is positioned on a wearer. The rear part 22 of the diaper hugs snugly to the rear part of the belt 42. The intermediate crotch part 24 of the diaper is drawn between the wearer's legs and rests snugly against the wearer's body. The belt is closed, being fastened by the belt closure devices 48A-48D. When this embodiment is adjusted properly, the front and rear fastening devices 30B, 28C, and 28D, shown in FIG. 7, rest on the wearer's hips.

FIG. 8 illustrates a front view of the diaper fully attached to the waist belt, which is positioned on a wearer. The front part 21 of the diaper hugs snugly to the front part 40 of the belt. The intermediate crotch part 24 of the diaper is drawn between the wearer's legs and rests snugly against the wearer's body. When this embodiment is adjusted properly, the front fastening devices 30A and 30B rest on the wearer's hips.

### Operation of Waist Belt and Absorbent Article

In operation, the diaper and waist belt garment work together to overcome some of the disadvantages of the prior art discussed above. The assembly is put onto the wearer in this embodiment by first securing the separate waist belt of FIGS. 3 and 4 onto the wearer's natural waist above the hips. The front part 40 of the waist belt is centered below the wearer's navel or bellybutton. The two halves of the rear part 42A and 42B are then brought around the wearer's back and secured by fastening the belt closure devices 48A-48D. The rear parts 42A, 42B should be centered at the wearer's spine above the buttocks.

The diaper shown in FIGS. 1 and 2 can then be attached in one embodiment to the waist belt by first fastening the front fastening devices 30A and 30B to the front fastening devices 44A, 44B of the belt, shown in FIGS. 3 and 4, respectively. Then the diaper is drawn between the wearer's legs and up above the wearer's buttocks. At this stage in the operation of this embodiment, the caregiver has several choices for fastening the rear part 22 of the diaper to the rear parts 42A, 42B of the belt. Fastening rear fastening devices 28A and 28D to rear fastening devices 46A and 46D, respectively, will result in the loosest possible fit. Fastening rear fastening devices 28A and 28D to rear fastening devices 46B and 46C, respectively, will result in a tighter fit. Fastening rear fastening devices 28B and 28C to rear fastening devices 46A and 46D, respectively, will result in an even tighter fit. Fastening rear fastening devices 28B and 28C to rear fastening devices 46B and 46C, respectively, will result in the tightest fit. The ability to adjust this embodiment to four different tightness levels offers a great advantage to caregivers of infants and children, as children tend to grow quickly during the diapering years. Although this embodiment provides four tightness levels, other embodiments could have more or less, depending on the age and size of the intended wearer. This would be achieved by adding or removing snaps from either the waist belt or the diaper or both.

Once the two chosen rear fastening devices of the diaper are fastened to the two chosen rear fastening devices of the belt, the diaper is fully secured to the wearer using four fastening devices total. This is advantageous over many prior art references mentioned in the background, which require five, six, or more total fastening devices for the diaper to be fully secured to the belt, an inconvenience to the caregiver.

Alternatively, in another embodiment the diaper can be secured to the belt after applying the belt to the wearer by fastening the chosen rear fastening devices first, and then drawing the diaper between the wearer's legs to fasten the front fastening devices. Alternatively, before the belt is placed around the wearer's body and while it is still open (belt closure devices 48A-48D being unfastened), the front fastening devices 30A and 30B can be fastened to the front fastening devices 44A, 44B of the belt. Then the waist belt can be wrapped around the wearer's body and the belt closure devices 48A-48D can be fastened in line with the wearer's spine. Then the diaper can be brought between the wearer's legs in order to fasten the rear fastening devices.

Alternatively, before the belt is placed around the wearer's body, the belt can be closed using the belt closure devices 48A-48D. Then the front fastening devices 30A and 30B can be fastened to the front fastening devices 44A, 44B of the belt and two of the rear fastening devices of the diaper can be fastened to two of the rear fastening devices of the belt, creating a pant-like shape. Then the combined garment can be pulled onto the wearer in the manner of a pull-up diaper pant. Alternatively, the belt can initially be placed around the wearer's waist backward, with the rear parts 42A and 42B being joined directly beneath the wearer's navel. Then the chosen rear fastening devices can be employed to attach the rear part 22 of the diaper to the rear parts 42A, 42B of the belt. Next, the wearer or caregiver can rotate the belt until the front part 40 of the belt is centered below the wearer's navel before pulling the front part 21 of the diaper between the wearer's legs to fasten the front fastening devices. The previous two methods both allow the wearer to be able to put on the diaper assembly by him or herself.

Other embodiments may have additional methods of attaching the garments to the wearer. The large number of ways to apply the garments to the wearer offers flexibility to the caregiver that most of the prior art lacks. When babies achieve mobility, many are not content to lie still in the dorsal recumbent position for the amount of time required to put on a traditional diaper. Being able to put the diaper on the child in any number of positions, including hands and knees, standing, while being held by the caregiver, and others, is an advantage of this embodiment. The ability for an older child or adult to put the diaper on by him or herself, granting the wearer independence from a caregiver, is also an advantage of this embodiment.

Once the diaper is on the wearer, the diaper can be removed and replaced with an identical second diaper by unfastening the front and rear fastening devices in any order to release the wet or soiled diaper. The waist belt remains in place during this process; the replacement diaper is attached to the waist belt in any one of the manners described above. Provided that the belt is not soiled with feces, the same belt can remain on the wearer for many diaper changes before needing to be washed. The wet or soiled diaper of this embodiment can also be washed and reused.

Since the belt remains on the wearer during diaper changes, removing the diaper of this embodiment is much quicker than with traditional diapers and much of the prior art. A child can remain standing, crawling, walking, cruising, or any number of other positions during the process. Putting the replacement diaper onto the child is also simpler than putting on the first diaper, as the belt is already in place; thus, the second diaper can be put onto the child while standing, crawling, cruising, among other positions. The caregiver can also both detach and replace the diaper with one hand while holding the child with the other. This is advantageous in situations such as a public restroom that does not have a changing pad available for the caregiver's use. This is also advantageous to caregivers who may be physically disabled.

The operation of this embodiment continues with offering the toilet to the wearer. Provided that the diaper is not wet or soiled, the diaper can be partially opened to allow the wearer access to the toilet. FIG. 5 illustrates how the diaper can be unfastened in the rear and pulled forward between the wearer's legs while remaining attached at the front. The wearer can then be placed on a toilet or other receptacle. With the diaper in this position, the caregiver can easily access the wearer's rear end for cleaning after a bowel movement. When the wearer is finished eliminating, the diaper can be brought back between the wearer's legs and refastened in the rear.

FIG. 6 illustrates how the embodiment can be unfastened in the front and pulled backward between the wearer's legs while remaining attached at the rear. The wearer can then be placed on a toilet or other receptacle. This position for the diaper is advantageous for male wearers, as the caregiver can easily access the wearer's penis to assist aiming into the toilet or other receptacle. The male wearer can also remain standing when the diaper is unfastened in the front, allowing him to stand while urinating. When the wearer is finished eliminating, the diaper can be brought back between the wearer's legs and refastened in the front. The ability to quickly and easily open the diaper from the front or the rear provides the caregiver with flexibility and is an advantage to this embodiment.

In the event that a caregiver is not satisfied with the absorbency of the diaper as it is shown in this embodiment, the caregiver has the option of adding absorbency via the pocket described above. Referring back to FIG. 1, the embodiment has a front elastic element 34 in the inner facing material 26 that is separate from the rear elastic element 36 in the liquid impervious top sheet 38. This creates a pocket into which additional absorbent units may be inserted, allowing the caregiver the flexibility to customize the absorbency of the garment for the needs of the specific wearer and circumstance. For instance, more absorbency may be desired as the child ages, during extended periods of wear such as naps and car rides, or due to the caregiver's preference.

### Alternative Embodiments

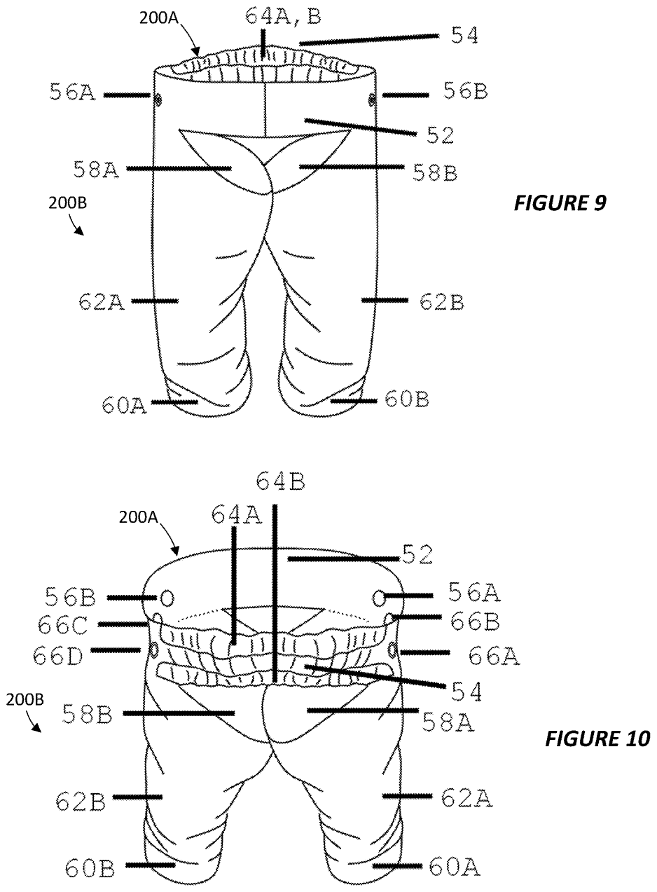

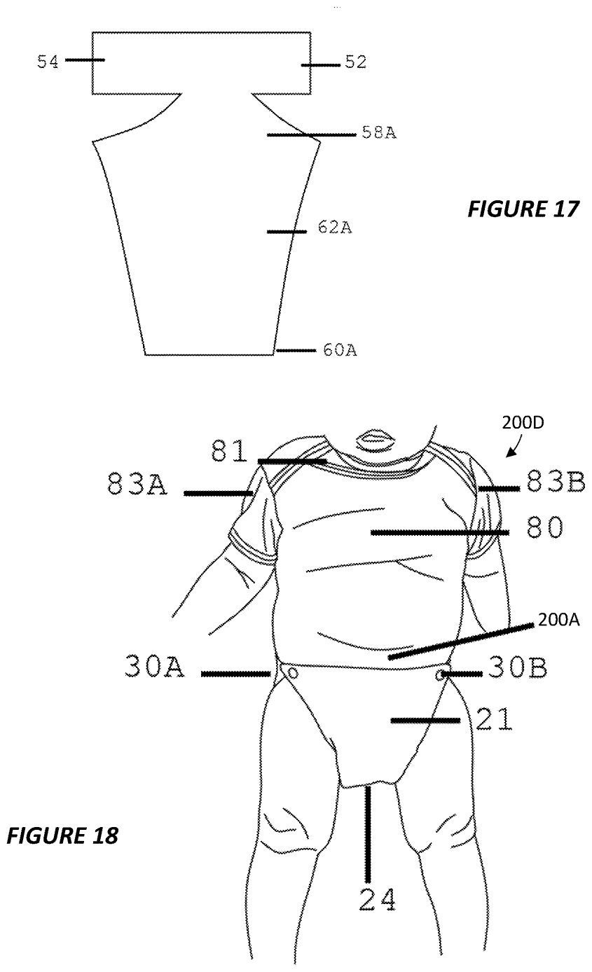

FIG. 9 shows a front view of a detached pant garment with a waist belt and FIG. 10 shows a rear view of the same detached pant garment. In this embodiment, the pant garment is comprised of fleece, but other materials for clothing, such as wool or suede, would be suitable. In this embodiment, the pant garment is shaped by folding a length of fleece in half and cutting two pattern shapes such as the one shown in FIG. 17. These two pattern shapes are sewn right sides together along front parts 52 and along rear parts 54 to form a continuous ring for the waist belt. The waist belt is then folded along its length and sewn in place in the same manner as folding and sewing the waist belt of FIG. 3. Finally, pant legs with upper leg holes 58A-58B, sleeves 62A, 62B for the legs, and lower foot holes 60A-60B, shown in FIGS. 9 and 10, can be shaped by sewing the lateral edges of each of the individual pattern shapes shown in FIG. 17 starting at the upper leg hole 58A and ending at the lower foot hole 60A. FIGS. 9 and 10 also show the elastic elements 64A and 64B, which are subsequently added to provide shaping.

Fastening devices can also be seen in FIGS. 9 and 10, with front fastening devices 56A, 56B of the pant garment being male snaps in this embodiment. In FIG. 10, rear fastening devices 66A-66D of the pant garment can also be seen, with the rear fastening devices being female snaps in this embodiment.

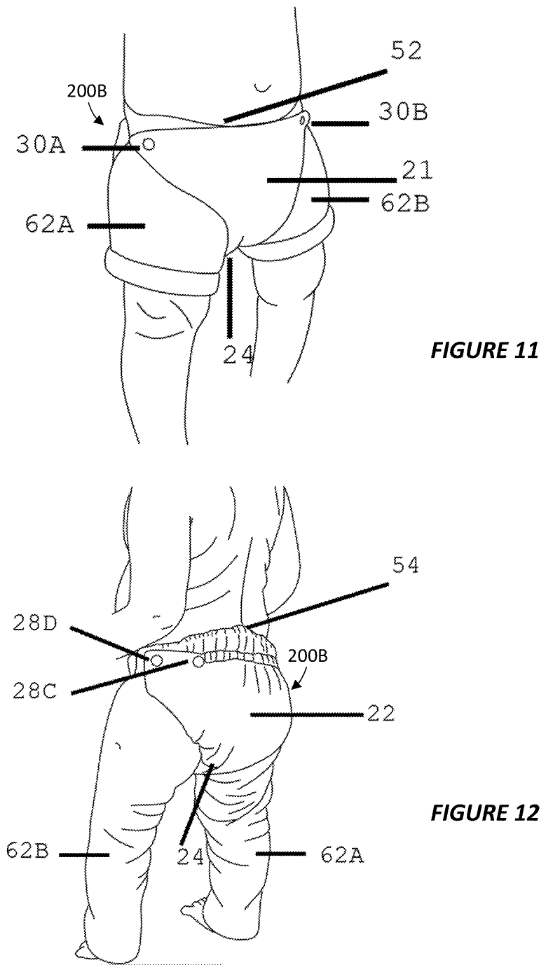

FIG. 11 illustrates a front view of the diaper fully attached to the pant garment, which is positioned on a wearer. The front part 21 of the diaper hugs snugly to the front part 52 of the pant garment. The intermediate crotch part 24 of the diaper is drawn between the wearer's legs and rests snugly against the wearer's body. When this embodiment is adjusted properly, the front fastening devices 30A and 30B rest on the wearer's hips. In this embodiment, the sleeves 62A, 62B for the legs are cut short enough to be worn as shorts ending just above the wearer's knees, although other lengths and styles could also be crafted.

FIG. 12 illustrates a rear view of the diaper fully attached to the pant garment, which is positioned on a wearer. The rear part 22 of the diaper hugs snugly to the rear part 54 of the pant garment. The intermediate crotch part 24 of the diaper is drawn between the wearer's legs and rests snugly against the wearer's body. When this embodiment is adjusted properly, the rear fastening devices 28C and 28D of the diaper, shown in FIG. 12, rest on the wearer's hips. In contrast to FIG. 11, this embodiment illustrates sleeves 62A, 62B for the legs that are cut long enough to cover the wearer's legs all the way to the heels of the wearer's feet.

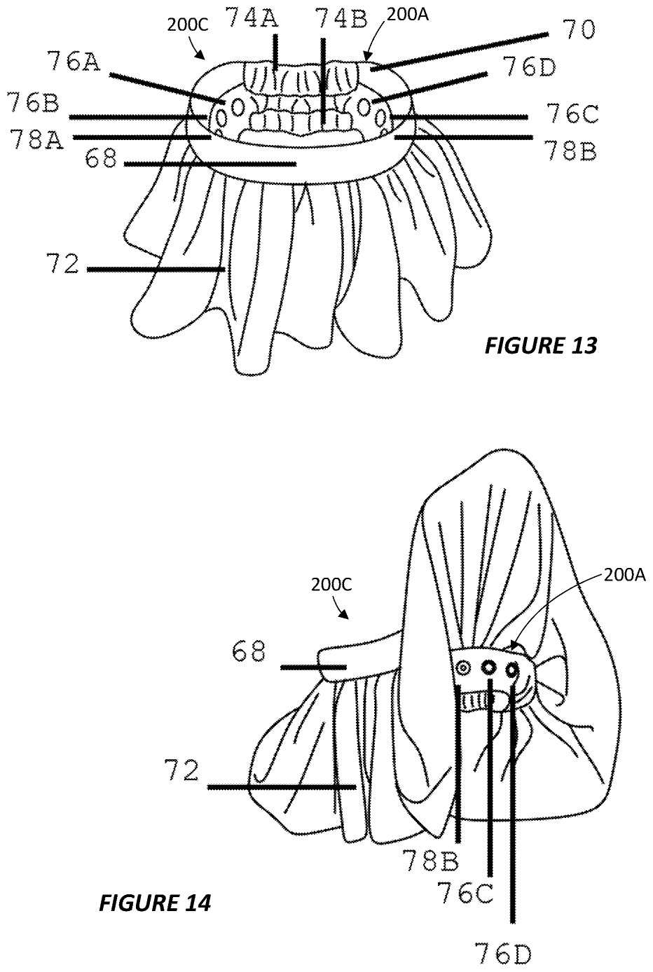

FIG. 13 illustrates a perspective view of a skirt garment with waist belt with a front part 68 and a rear part 70. In this embodiment, the waist belt of the skirt garment is comprised of fleece, but other materials such as wool or PUL could be used. The waist belt of the skirt is shaped by cutting a rectangle of fleece of dimensions 10.16 centimeters by 53.34 centimeters, although other sized rectangles would be appropriate for embodiments intended for larger or smaller individuals. The two lateral ends of this rectangle are sewn together to form a ring, with the seam in the rear part 70. A length of skirt material 72 is then cut and gathered around the waist belt of the skirt, which is then partially folded over the raw edge of the skirt material and sewn into place. The skirt fabric in this embodiment is made from a woven knit fabric, although other materials such as cotton or polyester blends would also be sufficient. Elastic elements 74A and 74B are then added to provide shaping in the rear part 70. Rear fastening devices 76A-76D and front fastening devices 78A and 78B are then added to the waist belt. In this embodiment, the fastening devices are applied to the waist belt in such a manner that the skirt material 72 hangs over them.

FIG. 14 provides an alternate view of the location of the fastening devices on the skirt garment, which in this embodiment are snaps located on each side of the skirt waist belt between the front part 68 and the rear part 70. FIG. 14 shows one side of the waist belt when the skirt material 72 is lifted up to reveal the concealed rear fastening devices 76C and 76D and front fastening device 78B. In this embodiment, the front fastening device 78B is a male snap and the rear fastening devices 76C and 76D are female snaps. Other fastening devices could also work.

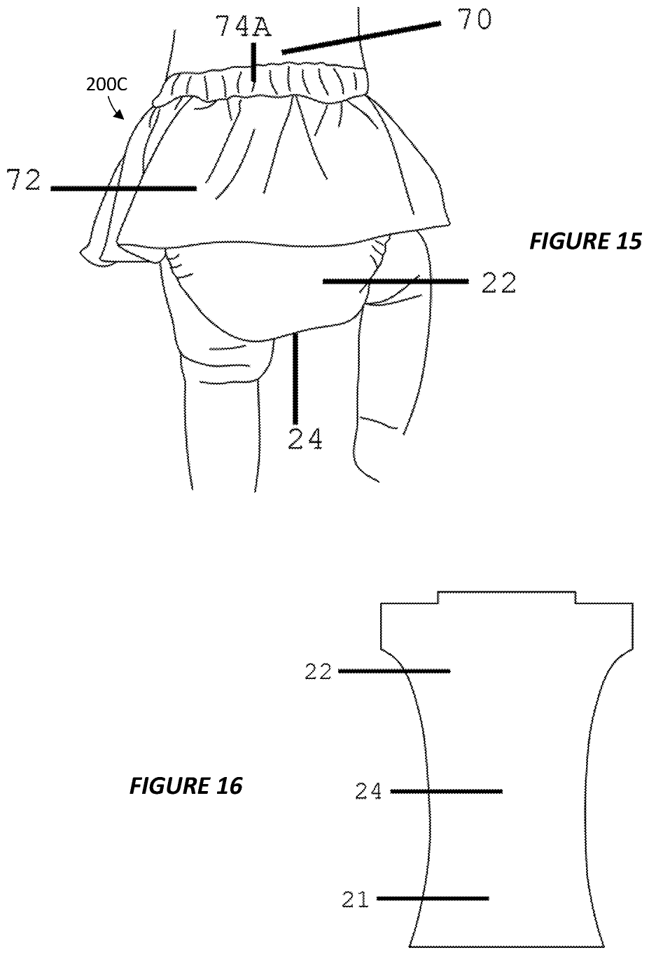

FIG. 15 illustrates a rear view of the diaper fully attached to the skirt garment, which is positioned on a wearer. The rear part 22 of the diaper hugs snugly to the rear part 70 of the waist belt of the skirt garment, but since the fastening devices are underneath the skirt material 72, the top portion of the rear part 22 of the diaper is hidden underneath the skirt material 72 along with all of the fastening devices. The intermediate crotch part 24 of the diaper is drawn between the wearer's legs and rests snugly against the wearer's body. Elastic element 74A is visible above the skirt material 72 in the rear part 70, but elastic element 74B is hidden beneath the skirt material 72.

FIG. 18 illustrates a front view of the diaper fully attached to a shirt garment with a waist belt 82, which is positioned on a wearer. The shirt garment is constructed in this embodiment by first fabricating a shirt 80 out of suitable material, for example cotton knit, polyester blend, fleece, or wool. The waist belt 82 is then attached to the lower raw edge of the shirt 80. This is accomplished in this embodiment by cutting a rectangle of fleece of dimensions 10.16 centimeters by 53.34 centimeters, although other sized rectangles would be appropriate for embodiments intended for larger or smaller individuals. The two lateral ends of this rectangle are sewn together to form a ring, which is then folded in half lengthwise with the lower raw edge of the shirt material sandwiched between the raw edges of the waist belt. Similar to the previously described garments, fastening devices and elastic elements are then added to the waist belt 82 of the shirt garment, although these are not shown in FIG. 18.

FIG. 18 illustrates how the shirt garment can be positioned on a wearer, with the diaper fully attached to the waist belt 82 of the shirt garment. The front part 21 of the diaper hugs snugly to the front part of the waist belt 82 of the shirt garment. The intermediate crotch part 24 of the diaper is drawn between the wearer's legs and rests snugly against the wearer's body. When this embodiment is adjusted properly, the front fastening devices 30A and 30B rest on the wearer's hips. In this embodiment, the shirt garment is crafted with short sleeves 83A, 83B for the arms; however, other embodiments could be sleeveless, have sleeves of varying lengths, have shoulder straps, among other styles. The wearer's head emerges through a neck hole 81.

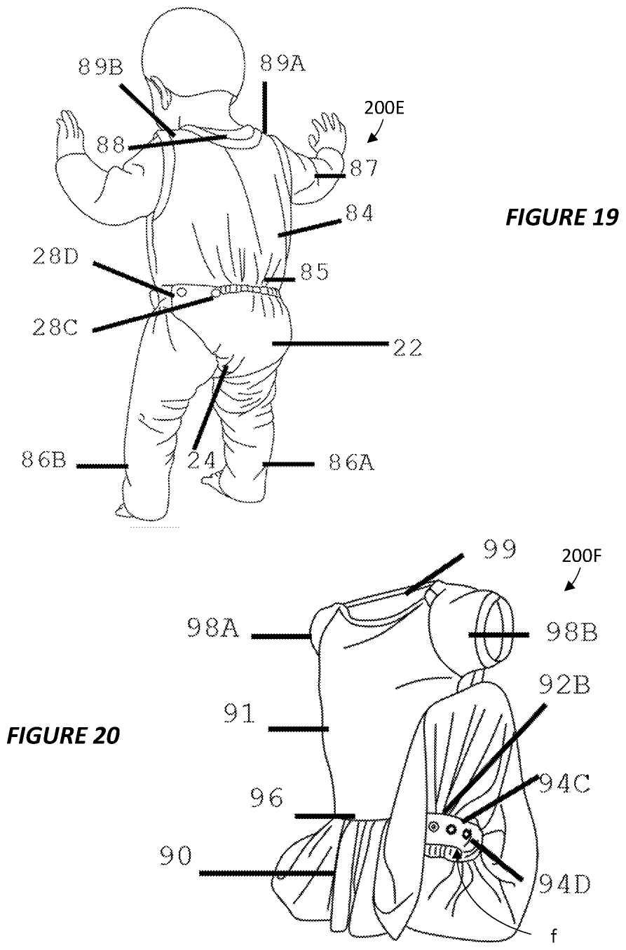

FIG. 19 illustrates a rear view of the diaper fully attached to a bodysuit garment with a waist belt 85, which is positioned on a wearer. The bodysuit garment has sleeves 86A, 86B for the legs as well as a shirt portion 84. This embodiment could be constructed by modifying the pattern for the pant garment shown in FIG. 17 by adding a shirt portion; fastening devices and elastic elements are then added in a similar fashion as described for the previously illustrated garments. Suitable materials for the bodysuit garment would be wool, fleece, or other fabrics used for clothing.

The wearer illustrated in FIG. 19 is wearing an undershirt 87 layered beneath the bodysuit garment, as the garment in this embodiment is sleeveless. Fastening devices 89A and 89B are added at the shoulders in this embodiment. These fastening devices 89A, 89B could be snaps, hook and loop, buttons, among other materials. Instead of being sleeveless, other embodiments could have attached sleeves of varying lengths or shoulder straps with fastening devices located at the wearer's chest.

The entire circumference of the upper portion of the waist belt 85 is attached to the entire lower edge of the shirt portion 84. Furthermore, the waist belt 85 is attached at the hips to the sleeves 86A, 86B for the legs, leaving the wearer's groin and buttocks exposed in a similar fashion to the pant garment described above. FIG. 19 illustrates how the exposed groin and buttocks are covered by the diaper when it is fully attached to the bodysuit garment. The rear part 22 of the diaper hugs snugly to the rear part of the waist belt 85 of the bodysuit garment. The intermediate crotch part 24 of the diaper is drawn between the wearer's legs and rests snugly against the wearer's body. When this embodiment is adjusted properly, the rear fastening devices 28C and 28D of the diaper, shown in FIG. 19, rest on the wearer's hips. This embodiment illustrates sleeves 86A, 86B for the legs that are cut long enough to cover the wearer's legs all the way to the heels of the wearer's feet; however, different lengths could be used for other embodiments, including embodiments with sleeves ending above the wearer's knees or with sleeves that have covers for the feet. The wearer's head emerges through a neck hole 88.

FIG. 20 illustrates a dress garment similar to the skirt garment described above. This embodiment could be constructed in a similar fashion as the skirt garment, with the additional step of crafting a shirt portion 91 that is attached to a waist belt 96 along with skirt material 90. Suitable materials for the shirt portion 91 and skirt material 90 would include cotton knit, polyester blends, fleece, wool, among others. This embodiment is illustrated with short sleeves 98A, 98B for the arms; however, other embodiments could be sleeveless, have sleeves of varying lengths, have shoulder straps, among other styles. A neck hole 99 is provided for the wearer's head to protrude through.

In FIG. 20, a portion of the skirt material 90 is shown as being lifted to reveal a front fastening device 92B, a male snap in this embodiment, and two rear fastening devices 94C and 94D, female snaps in this embodiment, located at the side of the waist belt 96. Although not pictured, a corresponding set of fastening devices are placed on the other side of the waist belt 96. When the skirt material 90 is allowed to hang freely, all of the fastening devices are concealed beneath the skirt material in this embodiment.

### Operation of Alternative Embodiments

In operation, the pant garment, skirt garment, shirt garment, bodysuit garment, dress garment, or any other embodiment of garment can replace the waist belt described in the operation of the waist belt and diaper above. For the pant garment, the wearer's legs are fed into the waist of the pant garment shown in FIG. 10; then the legs are fed into the individual upper leg holes 58A and 58B. The feet are fed through the sleeves 62A and 62B and come out the lower foot holes 60A and 60B. When the garment is correctly on the wearer, the waist belt of the pant garment will rest on the wearer's hips in the same way as the waist belt alone described above. The rear part 54 of the pant garment will be aligned with the wearer's spine and the front part 52 of the pant garment will be aligned with the wearer's navel. The wearer's buttocks and groin will be exposed until the diaper is fastened to the pant garment. The diaper can then be fastened to the waist belt of the pant garment by fastening the front fastening devices 30A and 30B of the diaper shown in FIG. 1 to the front fastening devices 56A, 56B of the pant garment, respectively, followed by a choice of rear fastening devices as described above. Alternatively, the rear fastening devices may be fastened first followed by the front fastening devices, or the diaper can be fully or partially fastened to the pant garment prior to applying the pant garment to the wearer and then pulled on like a pull-up, among other methods of application. The caregiver can then enjoy changing the wearer's diaper and offering the toilet to the wearer in the same manner described above for the waist belt. Unless it is soiled with feces, the pant garment can be worn for many diaper changes before needing to be removed and washed.

In operation of the skirt garment, the wearer's legs are fed into the waist of the skirt garment shown in FIG. 13. When the garment is correctly on the wearer, the waist belt of the skirt garment will rest on the wearer's hips in the same way as the waist belt alone described above. The rear part 70 of the skirt garment will be aligned with the wearer's spine and the front part 68 of the skirt garment will be aligned with the wearer's navel. The diaper can then be fastened to the waist belt of the skirt garment underneath the skirt material 72 by fastening the front fastening devices 30A and 30B of the diaper to the front fastening devices 78A, 78B of the skirt garment, respectively, followed by a choice of rear fastening devices as described above. Alternatively, the rear fastening devices may be fastened first followed by the front fastening devices, or the diaper can be fully or partially fastened to the skirt garment prior to applying the skirt garment to the wearer and then pulled on like a pull-up, among other methods of application. The caregiver can then enjoy changing the wearer's diaper and offering the toilet to the wearer in the same manner described above for the waist belt. Unless it is soiled with feces, the skirt garment can be worn for many diaper changes before needing to be removed and washed.

In operation of the shirt garment shown in FIG. 18, the wearer's head, torso, and arms are first fed into the waist belt 82 of the shirt garment prior to attaching the diaper. Next, the wearer's arms are fed through the sleeves 83A and 83B and the wearer's head is fed through the neck hole 81. The waist belt 82 is pulled downward until it rests on the wearer's hips.

In operation of the bodysuit garment shown in FIG. 19, the fastening devices 89A, 89B at the shoulders are first unfastened, thereby enlarging the neck hole 88 so that the wearer's legs followed by the torso can be inserted into the garment. The wearer's legs then continue downward through the waist belt 85 until they are fed individually into the sleeves 86A, 86B for the legs. In this embodiment, the feet are exposed at the bottom of the sleeves 86A and 86B, however, foot coverings could also be included. The waist belt 85 then rests at the wearer's hips. The shoulder fastening devices 89A and 89B are then fastened to secure the garment over the wearer's shoulders. The wearer's groin and buttocks are exposed at this stage of the operation.

In operation of the dress garment shown in FIG. 20, the wearer's head, torso, and arms are first fed into the waist belt 96 of the dress garment. Next, the wearer's arms are fed through the sleeves 98A and 98B and the wearer's head is fed through the neck hole 99. The waist belt 96 is pulled downward until it rests on the wearer's hips with the skirt material 90 hanging freely.

For each of the garments shown in FIGS. 18, 19, and 20 described above, the diaper can then be fastened to the waist belt of the garment by fastening the front fastening devices 30A and 30B of the diaper to front fastening devices of the garment followed by a choice of rear fastening devices as described above. Alternatively, the rear fastening devices may be fastened first followed by the front fastening devices, among other methods of application. The caregiver can then enjoy changing the wearer's diaper and offering the toilet to the wearer in the same manner described above for the waist belt. Unless they are soiled with feces, the shirt garment, bodysuit garment, and dress garment can all be worn for many diaper changes before needing to be removed and washed.

### Advantages

The above-described embodiment provides an advantage in that the absorbent article is compatible with many varieties of garments such as the waist belt, the pant garment, the skirt garment, the shirt garment, the bodysuit garment, and the dress garment described above, among other variations. Thus, the caregiver has the flexibility to choose between several different garments while still using the same diapers. The waist belt shown in FIG. 3 is a good choice for hot climates, hot seasons, for layering under traditional clothes or bodysuits, among other situations. The longer pant garment shown in FIG. 12 is a good choice for cold seasons, cold climates, among other situations. The shorter pant garment shown in FIG. 11 is a good choice for warm climates, warm seasons, for stylistic reasons, among other situations. The skirt garment shown in FIG. 15 is also a good stylistic choice for many different occasions or reasons.

Before an infant learns to walk, shirts and dresses that hang freely and do not snap or fasten beneath the groin tend to ride up the infant's torso while the infant is crawling, rolling, or being held by a caregiver. Traditional bodysuits, rompers, overalls, and other similar garments that fasten below the groin prevent the infant's navel and back from being exposed and are therefore a popular choice among caregivers. The embodiments illustrated in FIGS. 18, 19, and 20 all retain this advantage in addition to many of the other advantages discussed herein. The shirt garment shown in FIG. 18, for example, would be a good choice for keeping the wearer's torso covered in hot or warm climates or for layering under traditional pants, among other situations. The bodysuit garment shown in FIG. 19 would be a good choice for keeping the wearer's torso covered in cold seasons, cold climates, for bedtime or naps, among other situations. The dress garment shown in FIG. 20 would be a good stylistic choice for keeping the wearer's torso covered for many different occasions or reasons.

In this embodiment, the diaper, waist belt, pant garment, skirt garment, shirt garment, bodysuit garment, dress garment, and other variations of garment are all washable, reusable, adjustable, and compatible. Therefore, a caregiver could conceivably purchase a desired number of diapers along with a choice of waist belts, pant garments, skirt garments, shirt garments, bodysuit garments, dress garments, or other compatible garments and have all the equipment needed to enjoy the following benefits:

The caregiver will have enough diapers to last a significant portion of multiple wearers'diaper-wearing years, as the embodiment is washable, reusable, and can be tightened or loosened as the wearer grows.

The caregiver can quickly and easily attach the wearer's diaper without restricting the wearer's position, regardless of the garment of choice.

The caregiver can quickly and easily detach the wearer's diaper without removing the compatible garment and without restricting the wearer's position, regardless of the garment of choice.

The caregiver can quickly and easily change the wearer's diaper without removing the compatible garment and without restricting the wearer's position, regardless of the garment of choice.

The caregiver can offer the toilet or other receptacle to the wearer in a quick and efficient manner without removing the diaper or compatible garment from the wearer, regardless of the garment of choice.

The caregiver has a choice of opening the diaper from the front or the rear when offering the toilet or other receptacle to the wearer, regardless of the garment of choice.

The caregiver can tend to the wearer's needs in a variety of situations that are problematic for traditional diapers, such as in restrooms without changing pads or in the situation that the caregiver is disabled, regardless of the garment of choice.

The wearer can enjoy the independence of being able to put on and change the diaper without the aid of a caregiver.

The embodiment uses snaps instead of hook and loop closures, which are sturdier and hold up better to repeated washing.

The embodiment uses four fastening devices to successfully attach the diaper to the compatible garment, regardless of the garment of choice.

The embodiment can be applied to diaper-wearers of all descriptions, including infants, children, and adult incontinent individuals.

The embodiment's absorbency can be easily adjusted to suit the caregiver's and wearer's needs.

The caregiver can choose a compatible garment that will keep the wearer's torso covered while still enjoying the benefits listed above.

The benefits listed above will not be compromised by climate, season, or stylistic choices, as the diapers are compatible with multiple types of garments suitable for multiple situations and weather conditions.

While the above description contains many specificities, these should not be construed as limitations on the scope, but rather as an exemplification of several embodiments thereof. Many other variations are possible. For example, a garment with sleeves for the legs as well as a skirt would be one possible variation. Different lengths for the skirt are conceivable. Other types of garments with upper body coverings such as overalls or rompers could also be adapted to be compatible with the diapers shown in this embodiment. For embodiments with upper body coverings, many numbers of variations on the length and style for the arm sleeves are conceivable. A waist belt without belt closure devices that is always in the closed position of FIG. 4 is also conceivable. Both the diapers and the garments could be made in a variety of colors or patterns from a variety of materials. The diaper could also be made without a liquid impervious top sheet, transforming the diaper into a training pant or padded underwear; this would be an advantage to wearers who are transitioning from diapers to underpants. It is also conceivable to create disposable diapers that would be compatible with disposable or reusable belts and garments. Accordingly, the scope of the disclosure should be determined not by the embodiments illustrated, but by the appended claims and their legal equivalent.

What is claimed is:

1. A method of managing incontinence of a wearer, the method comprising:
providing an absorbent article comprising: a liquid-impervious outer material; an inner-facing material joined to the outer material to form an interior cavity; a first absorbent unit disposed within the interior cavity and configured to absorb liquids; a plurality of first fasteners disposed on an outward-facing surface of the absorbent article, wherein the absorbent article, when detached from the first carrier garment, contains no belt, no laterally-extending waistband wings, no waist-encircling structure, and no crotch panel, such that the absorbent article is incapable of being worn on a wearer without being fastened to a carrier garment;
providing a first carrier garment selected from the group consisting of: a waist belt, crotchless pants, crotchless shorts, a crotchless bodysuit, a skirt, a dress, and a shirt, the first carrier garment comprising: a hip-surrounding portion configured to encircle at least a portion of the hips or waist of the wearer; a plurality of second fasteners disposed on an outward-facing surface of the first carrier garment and configured to releasably engage the first fasteners of the absorbent article; a front part and a rear part, wherein the first carrier garment lacks any crotch panel extending between the front part and the rear part, such that the groin and buttocks of the wearer are fully uncovered and exposed when the first carrier garment is worn without the absorbent article, and wherein the first carrier garment is free of any absorbent material;
placing the first carrier garment onto the lower torso of the wearer such that the hip-surrounding portion encircles the hips of the wearer and the groin and buttocks of the wearer remain exposed;
after placing the first carrier garment onto the lower torso, removably connecting the absorbent article to the first carrier garment by engaging the first fasteners with the second fasteners such that: a front connection is formed between a front part of the absorbent article and the front part of the first carrier garment, and a rear connection is formed between a rear part of the absorbent article and the rear part of the first carrier garment,
wherein the front connection is formed by a first discrete set of mating fasteners located on a front region of the absorbent article and a front region of the first carrier garment, and the rear connection is formed by a second, separate discrete set of mating fasteners located on a rear region of the absorbent article and a rear region of the first carrier garment, such that the front connection can be released while the rear connection remains engaged, and the rear connection can be released while the front connection remains engaged, and
wherein, when the absorbent article is connected to the first carrier garment, the absorbent article is positioned outward of the first carrier garment such that at least a portion of the first carrier garment is sandwiched between the lower torso of the wearer and the absorbent article;
after removably connecting the absorbent article to the first carrier garment, disconnecting the absorbent article from the first carrier garment without removing the first carrier garment from the wearer; and
after disconnecting the absorbent article from the first carrier garment, removably connecting a replacement absorbent article to the first carrier garment while the first carrier garment remains continuously worn on the lower torso of the wearer and without removing the first carrier garment from the wearer's body, wherein the hip-surrounding portion of the first carrier garment remains encircling the wearer's hips throughout the disconnecting and reconnecting steps.

2. The method of claim 1, wherein disconnecting the absorbent article from the first carrier garment and connecting the replacement absorbent article to the first carrier garment are each performed while the first carrier garment remains continuously encircling the hips of the wearer, without removing the first carrier garment from the wearer, and without requiring the wearer to change from a seated, recumbent, or lying position to a standing position, and wherein the hip-surrounding portion of the first carrier garment remains continuously encircling the wearer's hips throughout both the disconnecting and reconnecting steps.

3. The method of claim 1, further comprising: after disconnecting the absorbent article from the first carrier garment, removably connecting the absorbent article or the replacement absorbent article to a second carrier garment that is a different type of carrier garment from the first carrier garment, wherein the second carrier garment is selected from the group consisting of: a waist belt, crotchless pants, crotchless shorts, a crotchless bodysuit, a skirt, a dress, and a shirt, wherein each of the first and second carrier garments lacks a crotch panel and each leaves the groin and buttocks of the wearer uncovered when worn without the absorbent article, and wherein the step of removably connecting to the second carrier garment is performed while the second carrier garment is already worn on the lower torso of the wearer.

4. The method of claim 3, wherein the first carrier garment is a waist belt and the second carrier garment is selected from the group consisting of crotchless pants, crotchless shorts, a crotchless bodysuit, a skirt, a dress, and a shirt.

5. The method of claim 1, wherein the absorbent article further comprises a second absorbent unit disposed within the interior cavity, wherein the first absorbent unit is configured to be positioned adjacent a front portion of the lower torso of the wearer and the second absorbent unit is configured to be positioned adjacent a rear portion of the lower torso of the wearer, and wherein the first absorbent unit and the second absorbent unit are each independently removable from and replaceable within the interior cavity without disassembling the absorbent article.

6. The method of claim 1, wherein the first fasteners and second fasteners comprise hook-and-loop fasteners.

7. The method of claim 1, wherein the first fasteners and second fasteners comprise snap fasteners.

8. The method of claim 1, wherein the absorbent article further comprises a top sheet disposed on the inner-facing material, wherein the top sheet is formed of a material that is liquid permeable and configured to contact the skin of the wearer.

9. The method of claim 1, wherein the absorbent article is reusable and machine washable, and wherein the plurality of first fasteners are snap fasteners comprising a socket component and a stud component that are each formed of a corrosion-resistant material configured to maintain fastening engagement after repeated machine washing cycles.

10. A garment system for managing incontinence of a wearer, the system comprising:
an absorbent article comprising: a liquid-impervious outer material; an inner-facing material joined to the outer material; a first absorbent unit disposed between the outer material and the inner-facing material and configured to absorb liquids from the wearer; a plurality of first fasteners disposed on an outward-facing surface of the absorbent article, wherein the absorbent article contains no structure configured to independently encircle the waist or hips of the wearer, and wherein the absorbent article, as a standalone component, contains no belt, no laterally-extending waistband wings, no waist-encircling structure, and no crotch panel, such that the absorbent article is incapable of being worn on a wearer without being fastened to a carrier garment;
a first carrier garment selected from the group consisting of: a waist belt, crotchless pants, crotchless shorts, a crotchless bodysuit, a skirt, a dress, and a shirt, the first carrier garment comprising: a hip-surrounding portion configured to encircle at least a portion of the hips or waist of the wearer and having an inward-facing side configured to contact the lower torso of the wearer and an outward-facing side configured to face away from the wearer; a front part and a rear part; a plurality of second fasteners disposed on the outward-facing side of the first carrier garment and configured to releasably engage the plurality of first fasteners, wherein the first carrier garment lacks any crotch panel extending between the front part and the rear part of the first carrier garment, such that the groin and buttocks of the wearer are structurally uncovered and exposed when the first carrier garment is worn without the absorbent article, and wherein the first carrier garment contains no absorbent material; and
wherein the first carrier garment and the absorbent article are configured such that, when the first and second fasteners are engaged: a front part of the absorbent article is connected to the front part of the first carrier garment to provide at least one front connection, and a rear part of the absorbent article is connected to the rear part of the first carrier garment to provide at least one rear connection,
wherein the front connection is formed by a first discrete set of mating fasteners located on a front region of the absorbent article and a front region of the first carrier garment, and the rear connection is formed by a second, spatially separate discrete set of mating fasteners located on a rear region of the absorbent article and a rear region of the first carrier garment, such that either connection may be released independently of the other, and
wherein the absorbent article is positioned outward of the first carrier garment such that at least a portion of the first carrier garment is sandwiched between the lower torso of the wearer and the absorbent article.

11. The garment system of claim 10, further comprising a second carrier garment that is a different type from the first carrier garment and is selected from the group consisting of: a waist belt, crotchless pants, crotchless shorts, a crotchless bodysuit, a skirt, a dress, and a shirt, wherein the second carrier garment comprises:
a hip-surrounding portion configured to encircle at least a portion of the hips or waist of the wearer and having an inward-facing side configured to contact the lower torso of the wearer and an outward-facing side configured to face away from the wearer; and
a plurality of third fasteners disposed on the outward-facing side of the second carrier garment and configured to releasably engage the plurality of first fasteners of the absorbent article,
wherein the second carrier garment, when worn without the absorbent article attached, is configured such that the groin and buttocks of the wearer are uncovered and exposed,
wherein the plurality of first fasteners of the absorbent article are configured to releasably engage both the plurality of second fasteners of the first carrier garment and the plurality of third fasteners of the second carrier garment such that the absorbent article is configured to be selectively and interchangeably transferred between the first carrier garment and the second carrier garment while the respective carrier garment remains worn by the wearer, and
wherein the second carrier garment lacks any crotch panel extending between its front and rear parts, and wherein neither the first nor the second carrier garment contains any absorbent material.

12. The garment system of claim 10, wherein the absorbent article further comprises a second absorbent unit disposed between the outer material and the inner-facing material, wherein the first absorbent unit is configured to be positioned adjacent a front portion of the lower torso of the wearer and the second absorbent unit is configured to be positioned adjacent a rear portion of the lower torso of the wearer.

13. The garment system of claim 10, wherein the first carrier garment is a waist belt.

14. The garment system of claim 10, wherein the first carrier garment is crotchless pants.

15. The garment system of claim 10, wherein the first carrier garment is a crotchless bodysuit.

16. The garment system of claim 10, wherein the first fasteners and second fasteners are selected from the group consisting of: hook-and-loop fasteners, snap fasteners, buttons, and magnetic fasteners.

17. The garment system of claim 10, wherein the absorbent article further comprises a top sheet disposed on the inner-facing material on an interior side of the absorbent article, wherein the top sheet is formed of a liquid-permeable material configured to contact skin of the wearer.

18. The garment system of claim 10, wherein the first carrier garment is selected from the group consisting of: a waist belt, crotchless pants, crotchless shorts, and a crotchless bodysuit, and wherein the first carrier garment consists of: the hip-surrounding portion, leg openings, and the plurality of second fasteners, with a crotch region being entirely absent such that the groin and buttocks of the wearer are fully exposed when the first carrier garment is worn without the absorbent article, and wherein the first carrier garment includes no crotch panel and no absorbent material.

19. A kit for managing incontinence, the kit comprising:
an absorbent article comprising: a liquid-impervious outer material; an inner-facing material joined to the outer material; at least one absorbent unit disposed between the outer material and the inner-facing material; a plurality of fasteners disposed on an outward-facing surface of the absorbent article, wherein the absorbent article, when standing alone, contains no structure configured to independently encircle the waist or hips of a wearer; and
two or more carrier garments, each carrier garment comprising: a hip-surrounding portion configured to encircle at least a portion of the hips or waist of the wearer; a plurality of carrier fasteners disposed on an outward-facing surface of the carrier garment and configured to releasably engage the plurality of fasteners of the absorbent article,
wherein each of the two or more carrier garments is a different type of garment selected from the group consisting of: a waist belt, crotchless pants, crotchless shorts, a crotchless bodysuit, a skirt, a dress, and a shirt,
wherein each of the two or more carrier garments lacks any crotch panel extending between its front and rear parts, such that the groin and buttocks of the wearer are structurally uncovered and exposed when any of the two or more carrier garments is worn without the absorbent article, and wherein none of the two or more carrier garments contains any absorbent material, and
wherein the absorbent article is configured to be removably connected to each of the two or more carrier garments interchangeably via the fasteners.

20. The kit of claim 19, wherein the absorbent article contains no belt, no waistband wings, no waist-encircling structure, and no crotch panel, and wherein the absorbent article is incapable of being worn on a wearer without being fastened to one of the two or more carrier garments.
